(19) United States
(12) Patent Application Publication
(10) Pub. No.: US 2009/0254132 A1
(43) Pub. Date: Oct. 8, 2009

(54) DEVICES AND METHODS FOR THE TREATMENT OF BONE FRACTURE

(76) Inventors: Robert M. Scribner, Boulder, CO (US); Lawrence R. Jones, Conifer, CO (US); Hansen A. Yuan, Fayetteville, NY (US)

Correspondence Address: Moore & Hansen, PLLP, 225 South Sixth Street, Suite 4850, Minneapolis, MN, 55402, US

(21) Appl. No.: 11/994,838

(22) PCT Filed: Jul. 7, 2006
(86) PCT No.: PCT/US06/26727

§ 371 Date: Nov. 10, 2008

Related U.S. Application Data
(60) Provisional application No. 60/697,260, filed on Jul. 7, 2005.

(51) Int. Cl. A61F 5/00

(52) U.S. Cl. 606/86 R

(57) ABSTRACT

Devices and methods for treating bones having bone marrow therein, or other targeted anatomical locations, including bones that are weakened, suffering from or prone to fracture and/or disease. The disclosed devices desirably prepare the targeted anatomical site for a flow of filling/stabilizing and/or therapeutic material, and then provide for control of the flow of material within the targeted anatomical site, measure the volume of material delivered to the site of interest, and prevent the placement of materials in unintended locations. Once material has been delivered, some or all of the flow control devices can be removed from the targeted anatomical site.

### CROSS-REFERENCE TO RELATED APPLICATION

This application claims the benefit of U.S. Provisional Patent Application Ser. No. 60/697,260, filed 7 Jul. 2005, entitled “Devices and Methods for the Treatment of Bone Fracture,” the disclosure of which is incorporated herein by reference.

### FIELD OF THE INVENTION

The present invention relates to devices and methods for treating bones suffering from fractures and/or diseases. More specifically, the present invention relates to devices and methods for repairing, reinforcing and/or treating the human spine and associated support structures using various devices, including osteotomy tools, and fill containment devices.

### BACKGROUND OF THE INVENTION

The healthy human spine is an intricate framework of bones and connective tissues which desirably supports the upper body and withstands the various physiological loads experienced by an individual during his or her normal daily activities. However, unusually high loading of the spine (such as trauma, repetitive heavy physical labor or the effects of sports or other intense physical activities), or loading of a weakened spine (where disease, neglect or medical treatment has reduced the strength of the bones and/or connective tissues to below the level necessary to withstand normal physiological loads—including osteoporosis, bone cancer, arthritis, various treatments causing elevated steroid levels, as well as the excessive use of alcohol and/or tobacco), can cause significant damage to the spinal anatomy. Such spinal damage can have extremely disastrous consequences, including death, paralysis, permanent disability, disfigurement and/or intense pain.

While current treatment regimens for damaged and/or weakened spinal bones and cushioning/connective tissues are improving, spinal surgery is still a very invasive procedure and causes significant trauma to the patient. According to generally accepted surgical practice, it is typically necessary to cut or otherwise distract (and generally further damage) the connective structures covering the spine itself in order to access the bones and supporting soft-tissue structures of the human spine. These connective structures, which are critical for proper spinal stability, cannot be immediately repaired once the surgery is completed, but rather often take months or even years (if ever) to heal. In fact, it is often the case that the surgical procedure itself will cause more harm and/or pain to the patient than the injury itself, which is why many patients prefer to live with existing spinal pain and injuries rather than go through the rigors and subsequent rehabilitation of a surgical procedure. Moreover, even where surgery is attempted and is successful, the patient will often suffer ill effects from the invasive surgical procedure for weeks or months, and may not regain their full strength for years, if ever.

Two surgical techniques have been developed in an attempt to treat fractured spinal bones in a minimally-invasive procedure. One of these techniques, vertebroplasty, involves the injection of a flowable reinforcing material, usually polymethylmethacrylate (PMMA—commonly known as bone cement), through an 11-gage spinal needle into an injured vertebral body. Shortly after cement injection, the liquid filling material polymerizes and increases in hardness, desirably supporting the vertebral body internally, alleviating pain and preventing further collapse of the injected vertebral body.

In a modification of the vertebroplasty procedure, the posture of the patient is preferentially aligned by the use of external cushions or bolsters applied to pelvis and shoulder regions of the supine patient. This anatomic position attempts to decrease the compression of the injured vertebral body prior to the vertebroplasty procedure.

Another technique for treating vertebral fractures, kyphoplasty, is a more recently developed modification to the vertebroplasty technique. In a kyphoplasty procedure (also known as balloon-assisted vertebroplasty), an expandable device is inserted inside the damaged vertebral body, and is then expanded within the bone. Desirably, this procedure creates a void within the bone that can be filled with bone cement or other load bearing material, rendering the fractured bone load-bearing. In effect, the procedure creates an internal “cast,” protecting the bone from further fracture and/or collapse.

A further technique for treating vertebral fractures is a more recently developed modification to the kyphoplasty technique. In the further modified procedure a curette is inserted to the balloon formed cavity. The curette is applied to the cancellous bone at the margins of the cavity to further fracture the cancellous bone. This fracture of cancellous bone allows further volume expansion of the balloon, or directional control of the placement of added balloon volume in the direction of the fracture formed by the curette. Desirably, this procedure creates a greater void within the bone that can be filled with bone cement or other load bearing material, rendering the fractured bone load-bearing. The curette fracture desirably allows greater restoration of normal vertebral anatomy.

While vertebroplasty and kyphoplasty have both been shown to reduce some pain associated with vertebral compression fractures, both of these procedures have proven inadequate to reliably and repeatedly restore vertebral body anatomy or treat the vast majority of spinal fractures, especially high velocity spinal fractures.

### DETAILED DESCRIPTION

The devices and methods of the invention are concerned with one or more of the following: reduction of fracture of the vertebral body, including an increase in height of the vertebral body to a position approximate to the prefracture state; stability of the fracture by placement of a stabilizing material including flowable materials which set to a hardened condition; and containment of the fill material within the vertebral body.

### Vertebral Body Access

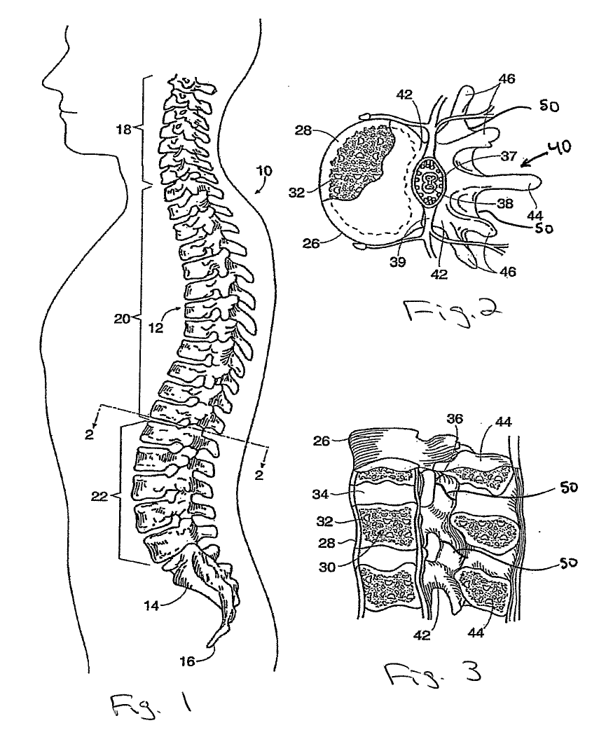

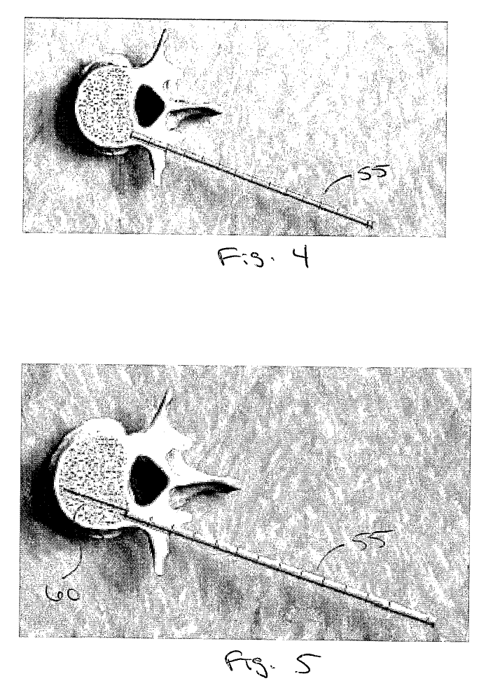

As FIGS. 1 to 3 show, each vertebra 12 includes a vertebral body 26, which extends on the anterior (i.e., front or chest) side of the vertebra 12. The vertebral body 26 is in the shape of an oval disk. The vertebral body 26 includes an exterior formed from compact cortical bone 28. The cortical bone 28 encloses an interior volume 30 of reticulated cancellous, or spongy, bone 32 (also called medullary bone or trabecular bone). A “cushion,” called an intervertebral disk 34, is located between the vertebral bodies 26.

An opening, called the vertebral foramen 36, is located on the posterior (i.e., back) side of each vertebra 12. The spinal ganglion 39 pass through the foramen 36. The spinal cord 38 passes through the spinal canal 37. The vertebral arch 40 surrounds the spinal canal 37. The pedicle 42 of the vertebral arch 40 adjoins the vertebral body 26. The spinous process 44 extends from the posterior of the vertebral arch 40, as do the left and right transverse processes 46.

Access to the vertebral body is typically accomplished by conventional transpedicular technique. The approach has been used for vertebral body biopsy and for access to the anterior vertebral body for reconstruction of trauma fracture of the anterior vertebral body.

Initial access to the vertebral body is obtained by an 11 gauge spinal needle, which perforates the skin and is advanced though the underlying muscle to contact the posterior surface of the pedicle under x-ray guidance. The center stylet of the needle is removed, and a k-wire is advanced through the lumen of the needle to the pedicle surface. The surgeon will place the k-wire to the pedicle guided by x-ray using the anterior-posterior (A-P) view. The k-wire is advanced across the pedicle to the anterior vertebral body with position monitored in the A-P and lateral views. Following advancement of the k-wire, the 11 gauge needle is removed leaving the k-wire in place.

A cannulated soft tissue dilator is then advanced over the k-wire to the surface of the pedicle. The dilator is intended to dilate or increase the diameter of the passage through the muscle and soft tissue. The dilator will be advanced across the pedicle to the posterior wall of the vertebral body when viewed using lateral x-ray.

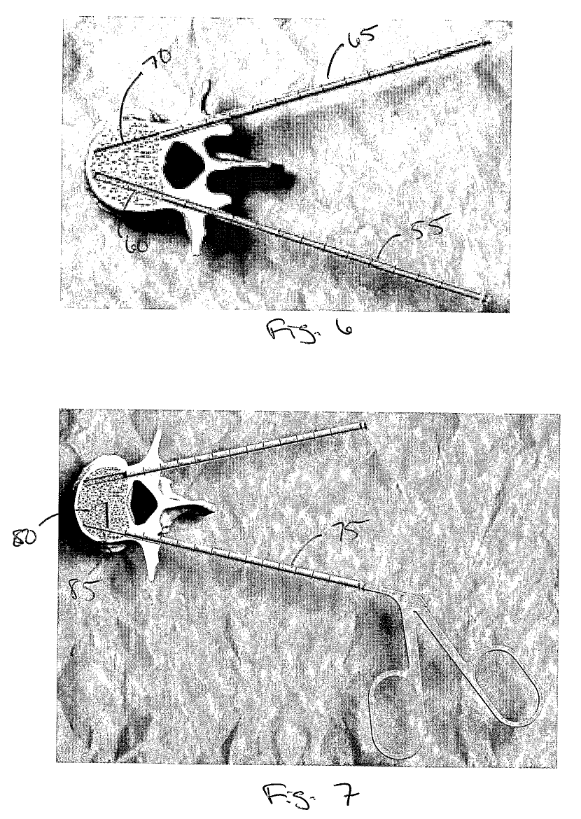

A cannula 55 is inserted over the dilator, and advanced to the posterior wall of the vertebral body when viewed using lateral x-ray. The dilator and k-wire are removed, leaving the cannula 55 in place to provide an access route to the vertebral body anterior of the posterior vertebral body wall. (FIG. 4.)

A twist drill may then be placed through the cannula to contact the cancellous bone within the anterior vertebral body. The drill is rotated and advanced though the cancellous bone to create a first passage (first linear passage) 60 though the cancellous bone for placement of osteotomy tools. The twist drill is removed, leaving the cannula in place to provide access to the first linear passage 60 in cancellous bone created by the twist drill. (FIG. 5.)

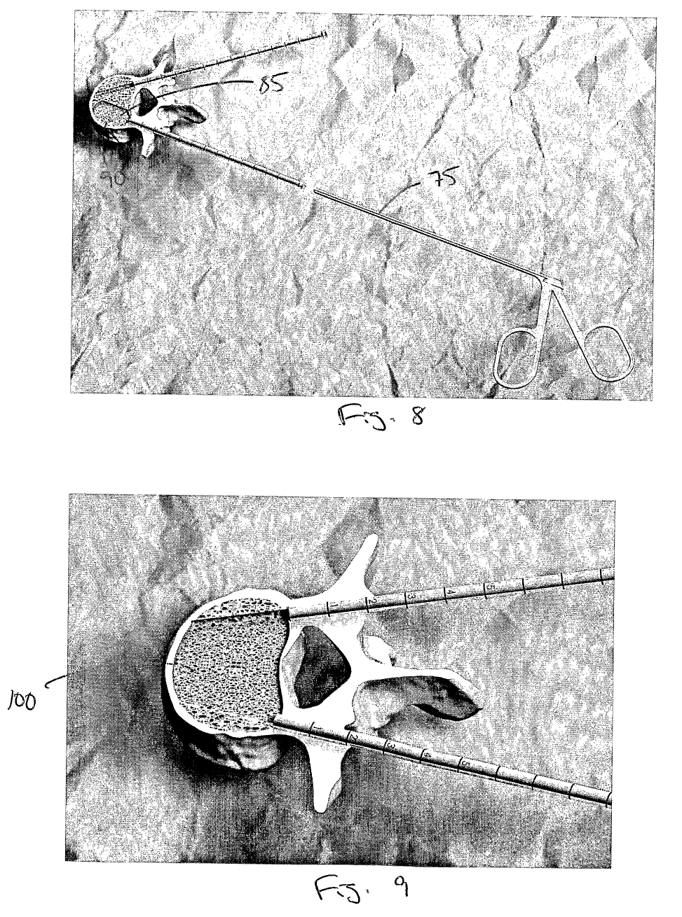

This procedure is then repeated on the second pedicle of the vertebral body, forming a second passage (second linear passage) 70 by means of the twist drill, and providing the surgeon with access routes to the anterior vertebral body by means of cannulae 55, 65 placed in both pedicles and the first and second linear passages 60, 70 formed within the vertebral body. (FIG. 6.)

Access to the vertebral body may also be accomplished by alternative anatomic placement of the instruments. Alternative access routes may include extrapedicular instrument placement, as in the thoracic spine, or posterolateral placement of the instruments avoiding placement within the pedicles of the vertebral body. These routes will provide access for formation of one or more linear passages within the cancellous bone.

### Osteotomy of the Vertebral Body

The osteotomy instrument 85 is placed through the cannula to the first linear passage in cancellous bone in the anterior vertebral body, the position monitored in lateral x-ray view.

By manual control of the surgeon, the blade of the osteotomy instrument is opened to contact cancellous bone at the margin of the first linear passage in bone created by the twist drill. Under x-ray view, the osteotomy instrument is advanced along the linear axis of the instrument to force the cutting blade to contact the cancellous bone. Contact of the blade in combination with linear motion will form a third passage (first lateral passage) 80 in the cancellous bone, formed in a lateral direction across the vertebral body. The blade of the osteotomy tool is progressively opened to advance in this first lateral passage 80 and maintain cancellous bone contact. Cyclical motion along the linear axis of the osteotomy tool moves the blade through the cancellous bone to enlarge the first lateral passage 80 by shear fracture of the cancellous bone. The position of the cutting blade is monitored in x-ray views to determine the advancement through cancellous bone, contact with cortical bone, and extent of formation of the first lateral passage 80 in the cancellous bone. (FIG. 7.)

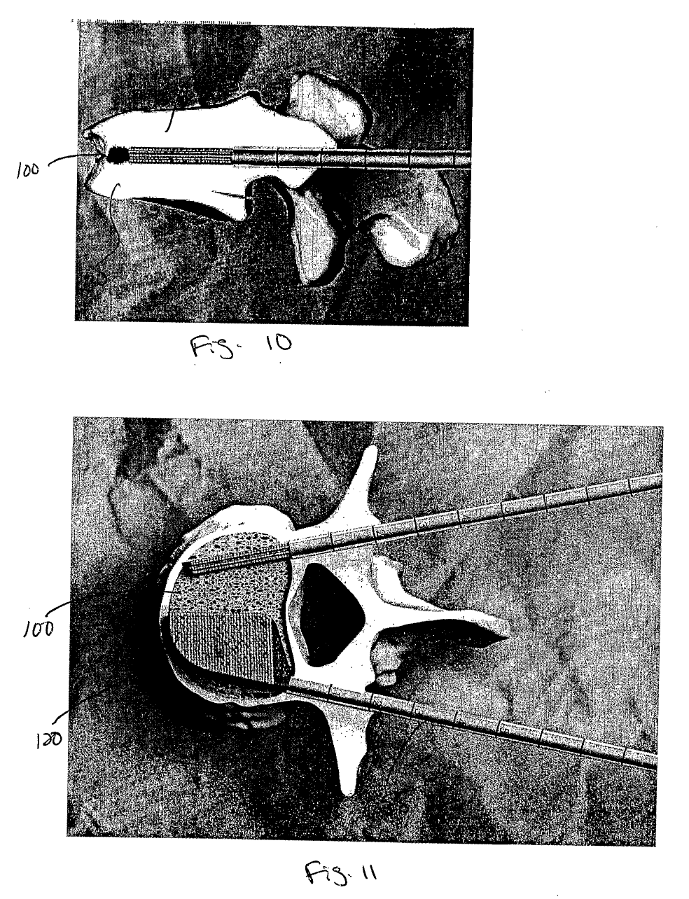

Following formation of the first lateral passage, the blade of the osteotomy instrument is moved to the original closed position. The osteotomy instrument is rotated 180 degrees within the first linear passage in bone. By manual control of the surgeon, the blade of the osteotomy instrument is opened to contact cancellous bone at the margin of the first linear passage in bone created by the twist drill. Under x-ray view, the osteotomy instrument is advanced along the linear axis of the instrument to force the cutting blade to contact the cancellous bone. Contact of the blade in combination with linear motion will form a fourth passage (first medial passage) 90 in the cancellous bone, formed in a medial direction across the vertebral body. The blade of the osteotomy tool is progressively opened to advance in the first medial passage and maintain cancellous bone contact. Cyclical motion along the linear axis of the osteotomy tool moves the blade through the cancellous bone to enlarge the first medial passage 90 by shear fracture of the cancellous bone. The position of the cutting blade is monitored in x-ray views to determine the advancement through cancellous bone and extent of formation of the first medial passage 90 in the cancellous bone. Following formation of the first medial passage 90, the osteotomy device is removed from the vertebral body. (FIG. 8.)

The above osteotomy procedure is repeated via the second pedicle of the vertebral body. The osteotomy instrument is placed through the second cannula to the second linear passage in cancellous bone in the anterior vertebral body, the position monitored in lateral x-ray view.

By manual control of the surgeon, the blade of the osteotomy instrument is opened to contact cancellous bone at the margin of the second passage in bone created by the twist drill. Under x-ray view, the osteotomy instrument is advanced along the linear axis of the instrument to force the cutting blade to contact the cancellous bone. Contact of the blade in combination with linear motion will form a fifth passage (second lateral passage) in the cancellous bone formed in a lateral direction across the vertebral body. The blade of the osteotomy tool is progressively opened to advance in this second lateral passage and maintain cancellous bone contact. Cyclical motion along the linear axis of the osteotomy tool moves the blade through the cancellous bone to enlarge the second lateral passage by shear fracture of the cancellous bone. The position of the cutting blade is monitored in x-ray views to determine the advancement through cancellous bone, contact with cortical bone, and extent of formation of the second lateral passage in the cancellous bone.

Following formation of the second lateral passage, the blade of the osteotomy instrument is moved to the original closed position. The osteotomy instrument is rotated 180 degrees within the second linear passage in bone. By manual control of the surgeon, the blade of the osteotomy instrument is opened to contact cancellous bone at the margin of the second linear passage in bone created by the twist drill. Under x-ray view, the osteotomy instrument is advanced along the linear axis of the instrument to force the cutting blade to contact the cancellous bone. Contact of the blade in combination with linear motion will form a sixth passage (second medial passage) in the cancellous bone, formed in a second medial direction across the vertebral body. The blade of the osteotomy tool is progressively opened to advance in the second medial passage and maintain cancellous bone contact. Cyclical motion along the linear axis of the osteotomy tool moves the blade through the cancellous bone to enlarge the second medial passage by shear fracture of the cancellous bone. The position of the cutting blade is monitored in x-ray views to determine the advancement through cancellous bone and extent of formation of the second medial passage in the cancellous bone. Following formation of the second medial passage, the osteotomy device is removed from the vertebral body.

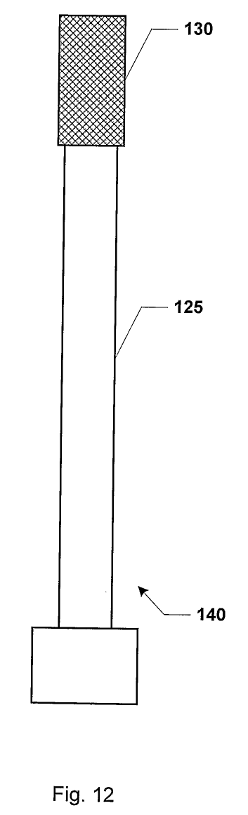

The second medial passage is formed until x-ray observation and measurement indicate that the second medial passage has made contact with the first medial passage, effectively forming by shear fracture an open plane (osteotomy plane) 100 within cancellous bone across the vertebral body, parallel and similar in configuration to the superior and inferior end plates of the vertebral body. The osteotomy plane within the vertebral body results from the combination of the multiple passages formed by means of the osteotomy tools, each passage of discrete dimension determined by the surgeon manipulation of the twist drill or osteotomy instruments. The osteotomy plane results in a separation of the vertebral body to two segments, the first (superior segment 105) superior to the osteotomy plane, the second (inferior segment 110) inferior to the osteotomy plane. (FIGS. 9-10.)

The formation of the lateral and medial passages in the cancellous bone is not limited to shear fracture by contact with a cutting blade. The passages may be formed by shear fracture of cancellous bone by means of a rotating blade, curette, preformed shapes of bladed instruments, abrasion of a traveling surface as with a band type saw, lateral translation of a rotating twist drill, or other methods developed by those skilled in the art.

The above method and devices do not require expansion of the first passage within the cancellous bone.

The formation of the lateral and medial passages within cancellous bone is accomplished by the shear fracture of cancellous bone in a single defined direction.

### Reduction of the Vertebral Body with Containment of Fill Material

Reduction of the vertebral body is accomplished by separation of the superior and inferior segments of the vertebral body along the osteotomy plane, moving the vertebral endplates to a greater separation distance and to a preferably more parallel alignment of the endplates relative to one another.

Reduction of the vertebral body is accomplished by the physical movement of the segments accomplished in combination with delivery of the stabilizing material to the osteotomy plane.

By means of the first access cannula, a vessel device 140 is used to deliver a vessel 130 within the osteotomy plane. The vessel device consists of an elongated catheter tubing 125 connected to the vessel 130, the vessel constructed of a non-expandable permeable or non-permeable membrane. The membrane material may be woven or non-woven, and is delivered to the osteotomy plane in a folded configuration of reduced profile.

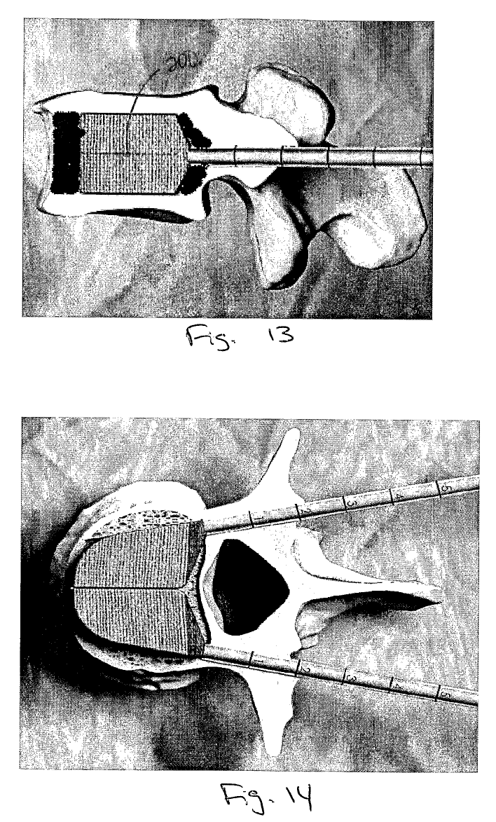

Using x-ray guidance, a radiopaque stabilizing material 120, 200 is delivered through the catheter tubing to the vessel. The hydrodynamic pressure of the filling material results in the unfolding of the vessel material as the volume of stabilizing material increases within the vessel. The hydrodynamic pressure of the filling material is applied across the membrane material to the cancellous bone, causing separation of the osteotomy plane 100 and an increase in the distance separating the inferior and superior segments of the vertebral body. Separation of the segments of the vertebral body results in the reduction of the vertebral body by increasing the vertebral body height to the prefracture state, and movement of the vertebral endplates to a more parallel configuration. (FIGS. 11, 13-14.)

Separation of the vertebral segments may also be achieved by delivery of granular solid materials to the vessel, such that the volume of granular material results in the unfolding of the vessel material as the volume of granular stabilizing material increases within the vessel. The mechanical pressure of the granular filling material is applied across the membrane material to the cancellous bone, causing separation of the osteotomy plane and an increase in the distance separating the inferior and superior segments of the vertebral body.

Separation of the vertebral segments may also be achieved by use of alternate means, such as the expansion of an inflatable device in contact with the cancellous bone surfaces of the osteotomy plane, including balloon type devices. The mechanical pressure of the inflatable device is applied to the cancellous bone, causing separation of the osteotomy plane and an increase in the distance separating the inferior and superior segments of the vertebral body.

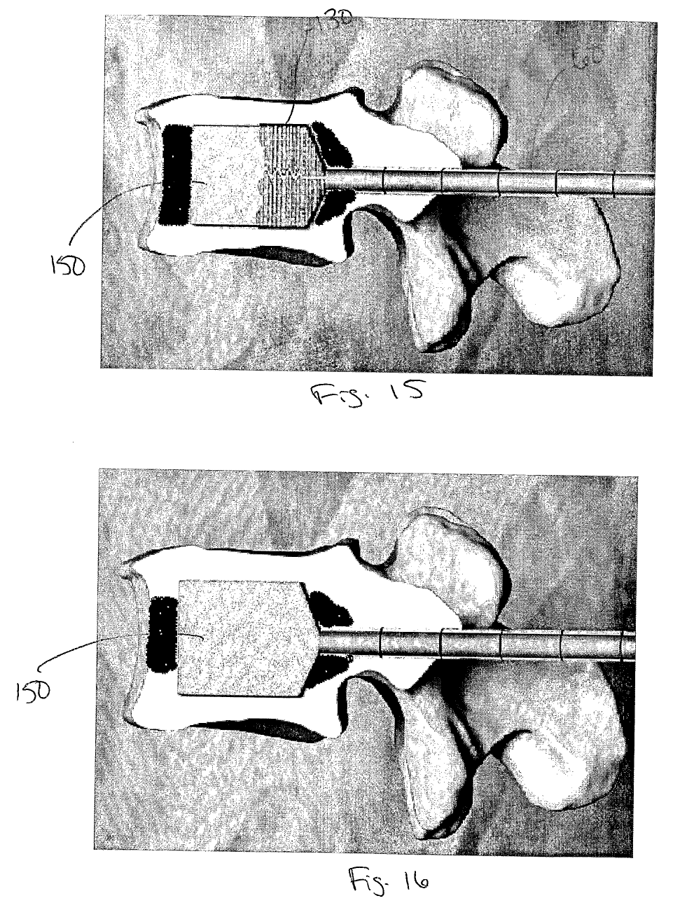

Reduction of the vertebral body is monitored by the surgeon observing the placement of the stabilizing material by x-ray. When reduction has been achieved, the delivery of additional volume of stabilizing material is terminated. The vessel 130 is opened to the osteotomy plane along a releasable opening in the membrane. The vessel is then withdrawn through the access cannula. The reduced diameter of the access cannula relative to the volume of delivered stabilizing material 150 results in the retention of the stabilizing material within the osteotomy plane as the vessel is withdrawn from the vertebral body. (FIGS. 15-16.)

Stabilizing material is retained with in the osteotomy plane by the soft tissues surrounding the vertebral body, including the anterior ligaments, posterior ligaments, cartilage, and muscular tissue. Flowable stabilizing material will set to a hardened condition in contact with and by interdigitation to the cancellous bone of the vertebral body, providing structural stability post reduction. Granular stabilizing materials such as calcium phosphates, calcium sulfates, autograft or allograft bone or other suitable materials will remain in contact with cancellous bone where bone remodeling will result in fracture stability.

Reduction of the vertebral body is accomplished by delivery of stabilizing materials to the osteotomy plane resulting from the formation of multiple passages within cancellous bone.

Reduction of the vertebral body results from the delivery of stabilizing materials to a position in contact with and within the cancellous bone of the vertebral body.

We claim:

1. A method of delivering a flowable material to a targeted anatomical site, the method comprising:
creating a lumen within the targeted anatomical site,
introducing a flow influencing device into the targeted anatomical site,
introducing the flowable material into the targeted anatomical site, and
removing the flow influencing device from the targeted anatomical site while leaving substantially all of the flowable material in the targeted anatomical site.

2. The method of claim 1, in which the flow influencing device comprises a vessel capable of containing the flowable material within the targeted anatomical site.

3. The method of claim 2, in which the vessel is sized and configured to pass through a cannular access path into the targeted anatomical site when the vessel is in a collapsed configuration.

4. The method of claim 2, in which the vessel comprises a vessel that can increase in volume within the targeted anatomical site.

5. The method of claim 4, in which the vessel comprises an opening that can be selectively opened.

6. The method of claim 5, in which the opening comprises a frangible opening.

7. The method of claim 6, in which the frangible opening is located at a distal portion of the vessel.

8. The method of claim 1, in which the flow influencing device comprises a vessel capable of containing the flowable material within the targeted anatomical site when releasably closed.

9. The method of claim 1, in which the flowable material is capable of achieving a less-flowable condition within the targeted anatomical site.

10. The method of claim 1, in which the targeted anatomical site is a bone.

11. The method of claim 10, in which the bone is a bone having bone marrow therein.

12. The method of claim 1, in which creating a lumen within the targeted anatomical site comprises creating a passage by compressing cancellous bone.

13. The method of claim 1, in which creating a lumen within the targeted anatomical site comprises creating a passage by cutting cancellous bone.

14. The method of claim 1, in which creating a lumen within the targeted anatomical site comprises creating a passage by manipulating cancellous bone.

15. The method of claim 1, in which creating a lumen within the targeted anatomical site comprises creating a passage by manipulating cortical bone.

16. The method of claim 1, in which creating a passage by compressing cancellous bone comprises expanding an expandable structure within cancellous bone.

17. The method of claim 1, in which the flowable material comprises bone cement.

18. The method of claim 1, in which the flowable material is capable of setting to a hardened condition within the targeted anatomical site.

19. The method of claim 1, in which introducing the flow influencing device into the targeted anatomical site comprises introducing the flow influencing device into the lumen.

20. A method of delivering a flowable bone cement to a bone having bone marrow therein, the method comprising:
creating a passage within the bone,
introducing a vessel in a collapsed configuration into the passage,
introducing the flowable bone cement into the vessel within the bone, and
removing the vessel from the bone while leaving at least a portion of the bone cement within the bone.

21. The method of claim 20, further comprising forming a second passage by cutting the bone marrow.

22. The method of claim 20, further comprising creating an opening in the vessel prior to removing the vessel from the bone.

23. A method for separating bone, comprising:
creating a first passage in a bone,
creating a second passage in the bone,
creating a first medial passage in the bone,
creating a second medial passage in the bone,
such that the first and second medial passages in the bone are joined to create a separation plane.
